(19) United States
(12) Patent Application Publication
(10) Pub. No.: US 2012/0142961 A1
(43) Pub. Date: Jun. 7, 2012

(54) METHOD FOR PRODUCING A RADIOPHARMACEUTICAL

(75) Inventors: Markus Kinzl, Dietzenbach (DE); Oliver Lade, Berlin (DE); Christan P. Schultz, Beverly, CA (US); Amo Steckenborn, Berlin (DE); Frank Thalmann, Muhlhausen (DE)

(73) Assignee: SIEMENS AKTIENGESELLSCHAFT, Munich (DE)

(21) Appl. No.: 13/138,814

(22) PCT Filed: Mar. 31, 2010
(86) PCT No.: PCT/EP2010/054316

§ 371 Date: Feb. 17, 2012

(30) Foreign Application Priority Data
Apr. 3, 2009 (DE) 10 2009 016 155.4
Jul. 29, 2009 (DE) 10 2009 035 647.9

(51) Int. Cl. C07C 51/15

(52) U.S. Cl. 562/423

(57) ABSTRACT

A method produces a radiopharmaceutical. In the method, an H—Li exchange is made by adding an alkyllithium to an isocyanide, wherein the α-H atom of the isocyanide is replaced with an Li atom. ¹¹CO₂ is added and bonded to the α-C atom of the isocyanide. By a two-stage hydrolysis, the Li atom is replaced with an H atom and an amino group is formed from the isocyanide group, for example, by adding NH₄Cl and HI. The reaction is continuously performed in particular in a microfluidic structure so that reaction times of less than 300 seconds can be achieved for the partial steps. Because the produced radiopharmaceutical has only a low half-life, the short production time has a positive effect on the yield of radioactive pharmaceutical.

### CROSS REFERENCE TO RELATED APPLICATIONS

This application is based on and hereby claims priority to International Application No. PCT/EP2010/054316 filed on Mar. 31, 2010 and German Application Nos. 10 2009 016 155.4 filed on Apr. 3, 2009 and 10 2009 035 647.9 filed on Jul. 29, 2011, the contents of which are hereby incorporated by reference.

### BACKGROUND

The invention relates to methods for producing a radiopharmaceutical or a precursor of a radiopharmaceutical which, as radioactive component, comprises at least one α-amino acid group labeled with the radionuclide ¹¹C.

An H—Li exchange is carried out by adding alkyllithium to an isocyanide where an isocyanide group is attached to an aliphatic C atom, wherein an α-H atom of the isocyanide is replaced by an Li atom. Furthermore, a carboxylation is carried out by adding ¹¹CO₂ and bonding it to the α-C atom of the isocyanide, where a C—C bond is formed while the Li atom remains at an O atom of the carboxyl group. A first hydrolysis where the Li atom is replaced by an H atom is then carried out, in particular by addition of NH₄Cl. A second hydrolysis where the amino group is formed from the isocyanide group is carried out, in particular by addition of HI.

A process of the type mentioned above has been described, for example by J. Bolster et al. in European Journal of Nuclear Medicine (1986) on pages 321-324 for producing tyrosine. Here, for carrying out the H—Li exchange butyllithium (abbreviated as BuLi) is added. Using the method described, it is also possible to prepare other amino acids in an analogous manner. Attention has to be paid to the fact that the individual reaction steps have to be carried out at different temperatures, thus requiring a change in temperature between the reaction steps. Moreover, some of the reactions generate heat which has to be dissipated. To prevent an instant liberation of the entire heat of reaction, the BuLi addition, for example, is carried out dropwise over a long period of, for example, 10 minutes. Owing to the temperature-dependent instability of the intermediate obtained, the carboxylation step furthermore requires very low temperatures as low as −100° C. since the intermediate has to be kept stable for several minutes. Accordingly, the great temperature differences required render the practice of the process relatively expensive. Moreover, the heating and cooling times required and the typically drop-wise addition of the BuLi prolong the course of the process. On the other hand, the process described produces a radioactive reaction product, and ¹¹C, having a half-life of about 20 min, decomposes relatively quickly, so that the radiopharmaceutical produced can only be used for a very limited time.

### SUMMARY

From what was said above, it is possible to derive the object of providing a method for producing a radiopharmaceutical having ¹¹C atoms, which method allows the production of a radiopharmaceutical having a relatively high radioactivity.

The inventors propose carrying out the H—Li exchange and the carboxylation in two partial steps directly one after the other, where these two partial steps are concluded within 300 seconds. By virtue of the relatively short residence time of the reaction intermediates in the partial steps mentioned, the decomposition of the radiopharmaceutical after the completion of its production has advantageously progressed to a relatively minor extent. As a result, the spatial distribution circle possible for the radiopharmaceutical can be widened in an advantageous manner. On the other hand, it is also possible to make do with smaller amounts of the radiopharmaceutical since its radioactivity will be more pronounced. Accordingly, the proposed method accelerates the time-critical partial steps of the H—Li exchange and the carboxylation to such an extent that the radioactive yield of the method can be maximized. Particularly preferably, the duration of the two partial steps mentioned is reduced to values of at most 120 seconds.

The reduction of the reaction times has the added advantage that the unstable intermediate obtained after the H—Li exchange requires less cooling because it can be made available immediately to the subsequent carboxylation reaction. This has the advantage that, by this process step, the reaction times can advantageously be reduced further to achieve the reaction times required for the two partial steps mentioned.

In accordance with particular embodiments, it is very advantageous to reduce the amounts of substance involved in the reactions in order to be able to carry out the two partial steps in relatively quick succession. Thus, in a batch process it is advantageous to carry out the two partial steps of the H—Li exchange and the carboxylation in a reaction space having a volume of less than 1 ml, preferably less than 500 μl. In such a small reaction volume, the heat generated during the reaction can advantageously be dissipated reliably and quickly even if (for example) the BuLi addition is instantaneous or takes place at most within a period of 5 seconds. The temperature change required between the two partial steps mentioned of the reaction can also be carried out more quickly.

It is particularly advantageous to carry out the two partial steps mentioned (H—Li exchange and carboxylation) by a continuous reaction in a channel structure into which chemicals involved in the partial steps are fed in continuously or quasi-continuously. Here, a plug flow process is carried out, i.e. the channel structure has a cross section which is sufficiently small so that back mixing of the reaction fluid passed through thus only occurs in a small amount, if at all, and a narrow residence time distribution is obtained. The reaction fluid is passed through a reaction channel where the reactions take place in each case starting at a point at which new chemicals are fed in the continuous course of the channel, with a continuous flow of reaction fluid under pre-set conditions. Here, the chemicals may be fed into the reaction fluid flowing past in an actually continuous manner at a constant volume flow, or quasi-continuously, i.e. in a quick succession of defined partial volumes.

Good results with respect to a continuous practice of the reaction can be achieved using channel structures having a channel diameter or edge lengths of the channel cross section (for a rectangular channel cross section) of less than 6 mm. Particularly preferably, the channel structure is designed as a microfluidic system. For the purpose of this discussion, a microfluidic system is to be understood as meaning a channel structure having a channel diameter or edge length of the channel cross section of less than 1 mm.

To further reduce the reaction times required, in an advantageous manner, it is envisaged that the chemicals involved in the partial steps are each added in succession or at least partially simultaneously in a continuous manner, but in each case within at most 5 seconds. Owing to the comparatively short feed-in times, it is advantageously possible also to conclude the reactions involved in the process more quickly. Addition over certain periods of time is possible firstly in a batch process and secondly with quasi-continuous addition in a quasi-continuous process of the chemicals, since the heat of reaction generated can be dissipated without any problems owing to the surface-to-volume ratio, which is favorable by virtue of the small channel cross sections.

In an advantageous manner, it is also possible to add the ¹¹CO₂ even before or during the H—Li exchange step. In this manner, advantageously, it is possible to save mixing times because the mixing-in of the ¹¹CO₂ is concluded at least substantially even when the H—Li exchange has finished, and the subsequent carboxylation partial step can thus be started immediately. This, too, may shorten the reaction time in an advantageous manner.

Another embodiment provides for the first and/or the second hydrolysis to be carried out continuously. In these partial steps of the overall reaction, too, it is possible to reduce reaction times in an advantageous manner. Moreover, the continuous practice of the hydrolysis is particularly advantageous, even if the preceding partial steps of H—Li exchange and carboxylation are already carried out continuously. This is because it is possible in this case for the partial steps of the hydrolysis to be integrated into the channel structure provided and simply be connected in series in the course of the reaction channel. Here, it is particularly advantageous for the first and/or the second hydrolysis to follow in a channel structure having a channel diameter or edge lengths of the channel cross section of less than 6 mm, which channel structure is preferably designed as a microfluidic structure.

The advantages associated therewith have already been explained further above.

Furthermore, it is advantageous for at least the two partial steps of the H—Li exchange and the carboxylation, preferably also the two partial steps of the first hydrolysis and the second hydrolysis, to be carried out under, compared to atmospheric pressure, elevated pressure (preferably at a pressure of up to 5 bar absolute). Hereby, it is possible to accelerate the reaction rate in an advantageous manner.

It is also advantageous for the ¹¹CO₂ to be pre-dissolved in a solvent and then added. In this manner, the mixing of the reaction fluid with the ¹¹CO₂ can be accelerated in an advantageous manner, which allows other reaction times to be reduced.

If the first and the second hydrolysis are carried out in one step, the reaction times are likewise reduced in an advantageous manner. Advantageously, reaction times of at most 120 seconds for the first hydrolysis and reaction times of at most 10 minutes for the partial step of the second hydrolysis can be achieved. Here, the second hydrolysis can be carried out advantageously at temperatures between 50 and 150° C. Here, too, the heating times are advantageously relatively short by virtue of the small reaction volumes.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 shows a working example of the reaction proceeding in accordance with the proposed method, and

FIG. 2 shows a microfluidic channel structure suitable for carrying out a working example of the proposed method.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

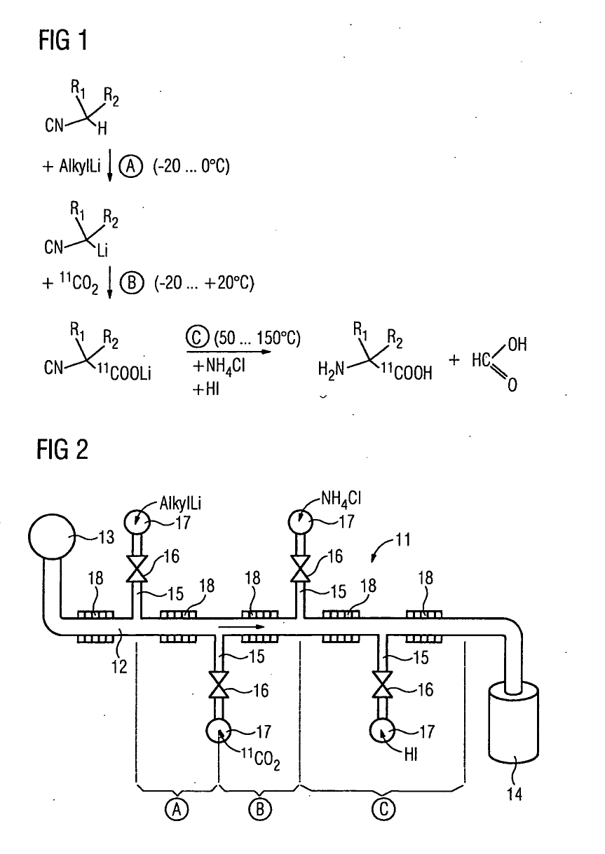

FIG. 1 shows a first partial step A of the H—Li exchange, a second partial step B of the carboxylation and a third partial step C which comprises a first hydrolysis and a second hydrolysis, of a precursor of the radiopharmaceutical formed by the radicals R₁ and R₂. Partial step A is carried out by adding alkyllithium, for example butyllithium (BuLi).

However, the crucial step is the carboxylation, i.e. the reaction with ¹¹CO₂, which takes place in partial step B. Here, ¹¹CO₂ is attached to the α-C atom of the isocyanide such that a C—C bond is formed. The Li atom remains at the O atom of the carboxyl group.

In the subsequent hydrolysis, in one step, NH₄Cl is added, which results in the replacement of the Li atom by an H atom, and HI is added, which results in the conversion of the isocyanide group into the amino group.

As can be seen in FIG. 1, compared to the related art described above, the temperature difference required from partial step A to partial step C can be reduced in an advantageous manner. Partial step A requires temperatures of from −20 to 0° C. Partial step B can be carried out at temperatures of from −20 to +20° C., which means that not all the heat produced in this partial step has to be dissipated. In particular, cooling of the intermediate from partial step A to very low temperatures is not required. In the subsequent step, in order to carry out the hydrolysis, the reaction fluid has to be brought to temperatures of from 50 to 150° C.

In the examples below, natural CO₂ was used instead of ¹¹CO₂ to demonstrate the reaction times which can be achieved. However, since, chemically, this is an identical substance, the values determined can be applied without restriction to the use of ¹¹CO₂.

### Example 1

Phenylglycin Ph-CH(CO₂H)—NH₂ was prepared from benzyl isocyanide Ph-CH₂—NC as starting material via the following reaction sequence. Here, Ph denotes the phenyl group C₆H₅. Bu denotes the butyl group C₄H₉.

Ph-CH₂—NC+BuLi→Ph-CHLi—NC (I a)

Ph-CHLi—NC+CO₂→Ph-CH(CO₂Li)—NC (I b)

Ph-CH—(CO₂Li)—NC+NH₄Cl→Ph-CH(CO₂H)—NC+NH₃+LiCl (II)

Ph-CH(CO₂H)—NC+2H₂O→Ph-CH(CO₂H)—NH₂+HCO₂H (catalyst: HI) (III)

The stable intermediate Ph-CH(CO₂H)—NC was prepared via the unstable intermediate Ph-CHLi—NC. To minimize the residence time of the unstable intermediate Ph-CHLi—NC, the partial steps H—Li exchange (I a) and carboxylation (I b) were carried out continuously in a channel structure in two microreactors having channel edge lengths between 0.5 and 5 mm, one reactor following directly after another. Here, 0.04 M benzyl isocyanide Ph-CH₂—NC (Aldrich Ltd, order number 133299) in tetrahydrofuran (Sigma Aldrich, order number 34946), 1.6 m butyllithium BuLi in hexane (Acros, order number 181278000) and gaseous CO₂ (Air Liquide, quality 4.5) were used. The BuLi was employed in a 3-fold molar excess, and the CO₂ in a 6-fold molar excess—in each case based on the benzyl isocyanide Ph-CH₂—NC. The reaction temperature for the partial steps H—Li exchange (I a) and carboxylation (I b) was in each case −20° C. The residence time in the microreactors including the connection channel to the second reactor or to the collection vessel for the stable intermediate Ph-CH(CO₂H)—NC was 60 s for the partial step H—Li exchange (I a) and 127 s for the partial step carboxylation (I b).

The first part of the hydrolysis (II) took place spontaneously in the collection vessel which was filled with 2% strength aqueous NH₄Cl solution. In the reaction vessel, samples were taken both from the aqueous and from the organic phase, and these samples were analyzed by HPLC. According to this analysis, 99.7% of the starting material had been converted after reaction step (II).

For the hydrolysis step in accordance with (III), both the aqueous and the organic phase of the collection vessel were mixed vigorously with 57% strength aqueous HI solution (Sigma Aldrich, order number 210013) in a 50 ml glass vessel and heated at 120° C. for 10 minutes. After cooling to room temperature, the mixture was neutralized using NaOH solution. Once more, samples were taken from both reaction mixtures and analyzed by HPLC. According to this analysis, the yield of phenylglycin Ph-CH(CO₂H)—NH₂ was 12.2%.

### Example 2

Phenylglycin Ph-CH(CO₂H)—NH₂ was prepared by a procedure as in Example 1. The BuLi was employed in a molar excess of 1.4 and the CO₂ in a molar excess of 4.4—in each case based on the benzyl isocyanide Ph-CH₂—NC.

The reaction temperature for the partial steps H—Li exchange (I a) and carboxylation (I b) was in each case 0° C. The residence time in the microreactors including the connection channel to the second reactor or to the collection vessel for the stable intermediate Ph-CH(CO₂H)—NC was 16 s for the partial step H—Li exchange (I a) and 33 s for the partial step carboxylation (I b). 93.8% of the starting material had been converted after reaction step (II). The yield of the phenylglycin Ph-CH(CO₂H)—NH₂ after the hydrolysis step (III) was 16.6%.

### Example 3

Phenylglycin Ph-CH(CO₂H)—NH₂ was prepared by a procedure as in Example 1. The BuLi was employed in a molar excess of 1.6 and the CO₂ in a molar excess of 5.5—in each case based on the benzyl isocyanide Ph-CH₂—NC. The reaction temperature for the partial steps H—Li exchange (I a) and carboxylation (I b) was in each case 0° C. The residence time in the microreactors including the connection channel to the second reactor or to the collection vessel for the stable intermediate Ph-CH(CO₂H)—NC was 33 s for the partial step H—Li exchange (I a) and 70 s for the partial step carboxylation (I b). 98.1% of the starting material had been converted after reaction step (II). The yield of the phenylglycin Ph-CH(CO₂H)—NH₂ after the hydrolysis step (III) was 30.8%.

FIG. 2 shows a microfluidic channel structure 11 which can be used to carry out the reaction shown in FIG. 1. The channel structure 11 has a reaction channel 12 through which the precursors of the radiopharmaceutical can flow in the direction of the arrow. They originate from a storage container 13 and flow into a collection vessel 14 for the finished radiopharmaceutical. Furthermore, feeds 15 are provided which can be used to feed in the chemicals shown in FIG. 1 via valves 16. The reaction steps A, B and C according to FIG. 1 are carried out in the sections of the reaction channel 12 marked in FIG. 2, allowing a continuous flow of the radiopharmaceutical formed and the chemicals fed in. The storage containers 17 for the chemicals to be fed in are each labeled with the chemicals according to FIG. 1. Also located at the reaction channel 12 are Peltier elements 18 which allow the temperature to be controlled over the length of reaction channel 12. Here, a control unit (not shown) with temperatures sensors (not shown) may be installed to monitor the process.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d1865 (Fed. Cir. 2004).

1-17. (canceled)

18. A method for producing a radiopharmaceutical or a precursor of a radiopharmaceutical which, as radioactive component, comprises at least one α-amino acid group labeled with a ¹¹C radionuclide, comprising:
carrying out an H—Li exchange by adding an alkyllithium to an isocyanide having the isocyanide group attached to an aliphatic C atom, the H—Li exchange replacing an α-H atom of the isocyanide with an Li atom;
carrying out a carboxylation by adding ¹¹CO₂ and bonding a carboxyl group ¹¹CO₂ to the α-C atom of the isocyanide, thereby forming a C—C bond while the Li atom remains at an O atom of the carboxyl group;
carrying out a first hydrolysis by addition of NH₄Cl, where the Li atom is replaced by an H atom; and
carrying out a second hydrolysis by addition of HI, where an amino group is formed from the isocyanide group, wherein
the H—Li exchange and the carboxylation are carried out in two partial steps directly after one another, and
the two partial steps of the H—Li exchange and the carboxylation are concluded within 300 seconds.

19. The method as claimed in claim 18, wherein
the two partial steps of the H—Li exchange and the carboxylation are concluded within 120 seconds.

20. The method as claimed in claim 18, wherein
each of the two partial steps of the H—Li exchange and the carboxylation is carried out at a temperature of from −20 to +20° C.

21. The method as claimed in claim 18, wherein
the two partial steps of the H—Li exchange and the carboxylation are carried out as batch process in a reaction space having a volume of less than 1 ml.

22. The method as claimed in claim 18, wherein
the two partial steps of the H—Li exchange and the carboxylation are carried out as batch process in a reaction space having a volume of less than 500 μl.

23. The method as claimed in claim 18, wherein
the two partial steps of the H—Li exchange and the carboxylation are carried out by a continuous reaction in a channel structure to which chemicals involved in the partial steps are fed in continuously or quasi-continuously.

24. The method as claimed in claim 23, wherein
the channel structure is rounded and has a channel diameter less than 6 mm or the channel structure is rectangular and has edge lengths of a channel cross section of less than 6 mm.

25. The method as claimed in claim 24, wherein
the channel structure is a microfluidic system having a channel diameter or edge lengths less than 1 mm.

26. The method as claimed in claim 18, wherein chemicals involved in the partial steps are added within at most 5 seconds of each other.

27. The method as claimed in claim 18, wherein
the ¹¹CO₂ is added before the H—Li exchange step is finished.

28. The method as claimed in claim 18, wherein at least one of the first and second hydrolysis is carried out continuously.

29. The method as claimed in claim 28, wherein
at least one of the first and second hydrolysis is carried out in a channel structure to which chemicals involved in the hydrolysis are fed,
the channel structure is rounded and has a channel diameter of less than 6 mm or the channel structure is rectangular and has edge lengths of less than 6 mm.

30. The method as claimed in claim 18, wherein
at least the two partial steps of the H—Li exchange and the carboxylation are carried out at an elevated pressure greater than standard atmospheric pressure and less than or equal to 5 bar absolute.

31. The method as claimed in claim 18, wherein the ¹¹CO₂ is pre-dissolved in a solvent and then added.

32. The method as claimed in claim 18, wherein the first and the second hydrolysis are carried out in one step.

33. The method as claimed in claim 18, wherein the first hydrolysis is concluded within 120 seconds.

34. The method as claimed in claim 18, wherein the second hydrolysis is concluded within 10 minutes.

35. The method as claimed in claim 18, wherein the second hydrolysis is carried out at a temperature between 50 and 150° C.
